(19) United States
(12) Patent Application Publication
(10) Pub. No.: US 2013/0072848 A1
(43) Pub. Date: Mar. 21, 2013

(54) Threaded lacrimal drainage tube method

(71) Applicant: Bruce B. Becker, Malibu, CA (US)

(72) Inventor: Bruce B. Becker, Malibu, CA (US)

(21) Appl. No.: 13/308,343

(22) Filed: Nov. 30, 2011

Related U.S. Application Data
(63) Continuation of application No. 11/916,780, filed on Jul. 7, 2008, now abandoned.
Application No. 11/916,780 is a continuation-in-part of application No. 11/145,847, filed on Jun. 6, 2005, now abandoned.

(51) Int. Cl. A61M 1/00

(52) U.S. Cl. 604/8

(57) ABSTRACT

A threaded lacrimal bypass drainage device uses a threaded cannula or tube for greater axial friction and adjustability. The tube has a keyed flange for engagement by a keyed screwdriver-type tool having a trocar coaxially engaging the tube and providing a distal cutting blade. A removable, coaxial, biocompatible washer discourages tissue overgrowth of the tube immediately after emplacement. One or more radial drainage holes formed near the distal end of the tube overcome axial blockages. Adjustments to the axial position of the tube can be readily made during successive office visits by the patient.

### PRIOR APPLICATION

This is a continuation of U.S. application Ser. No. 11/916,780 filed Jul. 7, 2008 which is a 371 of International Application Serial No. PCT/US2006/021692 filed May 6, 2006 designating the U.S. which is a continuation-in-part of U.S. application Ser. No. 11/145,847 filed Jun. 6, 2005.

### FIELD OF THE INVENTION

This invention relates to devices and methods for correcting drainage in the lacrimal system, and more particularly to addressing canalicular stenosis or obstruction, and nasolacrimal duct obstruction that does not respond to dacryocystorhinostomy or dilation.

### BACKGROUND

The orbital portion of the lacrimal gland is located in the superotemporal orbit and the palpebral portion of the lacrimal gland is located on the posterior surface of the superotemporal upper lid. The lacrimal gland produces the aqueous portion of the tear film. Ductules from the orbital portion of the lacrimal gland pass through the adjacent palpebral lacrimal gland to empty into the superior conjunctival cul-de-sac. Smaller accessory lacrimal glands in the upper and lower lids also contribute to tear production. The tears bathe the surface of the eye and then drain into the puncta and canaliculi in the medial upper and lower lids. The superior and inferior canaliculi join as the short common canaliculus. The tears flow from the superior and inferior canaliculi through the common canaliculus, into the lacrimal sac, and down the nasolacrimal duct into the nose.

The canaliculi can become obstructed or stenotic on a congenital basis, from trauma such as lacerations, from inflammation, or the obstruction can be idiopathic. When the upper and lower canaliculi or the common canaliculus become obstructed, tears can no longer drain from the surface of the eye through the lacrimal system into the nose. The tears well up in the eye as a result, and run down the face. The excess tears blur the vision and the patient has to constantly dab the eye.

The nasolacrimal duct can also become obstructed leading to tearing. Tears stagnate in the lacrimal sac and bacteria multiply, causing infection which can lead to painful enlargement of the lacrimal sac filled with pus, and discharge over the eye.

Canalicular obstruction or stenosis is usually treated by forming a new passage through the obstruction with a probe, and dilation with probes or a balloon catheter. A silicone tube is often placed as a temporary stent. At times a dacryocystorhinostomy (DCR) is also performed. A DCR consists of surgically creating anew passage from the lacrimal sac into the nose. This can be performed with a balloon catheter as disclosed in my U.S. Pat. Nos. 5,021,043 and 5,169,386, using an endoscope or externally through an incision.

Canalicular obstruction often recurs after dilation and silicone intubation. A new drainage system is then required to allow tears to drain from the conjunctival cul-de-sac into the nose. This necessitates placement of a permanent drainage tube, often called a canalicular bypass tube, which extends from the very medial conjunctiva into the nose. The tube is angled somewhat inferiorly to aid in tear drainage. A conjunctivodacryocystorhinstomy (CDCR), which is a DCR extending through the conjunctiva, is performed prior to or at the same time as tube emplacement.

A DCR for nasolacrimal duct obstruction without canalicular obstruction is usually successful. However, tearing persists in some patients in spite of a DCR that seems patent. The DCR cannot drain a large enough volume of tears in these patients, some of whom produce a larger volume of tears than normal. A canalicular bypass tube is often required in such patients.

The most commonly used canalicular bypass tube is a pyrex glass tube known as a “Jones tube” as described in Glatt, H. J. and Putterman, A. M., Conjunctivodacryocystorhinostomy in Mauriello, Jr., J. A. (Ed), Unfavorable Results of Eyelid and Lacrimal Surgery: Prevention and Management, Boston: Butterworth Heinemann, 2000; pp 577-582. It has a flange on the end that opens to the ocular surface. The end that is in the nasal cavity has no flange or a very minimal flange which is not adequate to discourage axial migration of the tube toward the eye. These tubes range from just over 2 millimeters (“mm”) to 2.4 mm in outside diameter and 13 mm to 22 mm in length. A less commonly used tube is made of polyethylene and is not as rigid as glass. It is cut to the desired length during surgery.

The tube is placed in the following manner. The medial conjunctiva is excised with a small scissors. A large diameter needle is pushed through the conjunctival opening, angled about 25 degrees inferiorly, into the nasal cavity. The nasal end is visualized to be sure that the location and angle are proper. The needle is withdrawn and a two-sided knife blade is brought through the same tract. The knife blade is withdrawn and the tract is further dilated with dilators or balloon catheters. Next, a narrow diameter oblong rigid metal probe is placed through the lumen of the tube. The probe is placed in the tract to act as a guide. The tube is then slid along the probe and pushed into the tract so that it extends from just lateral to the conjunctiva through the tract into the nasal cavity.

Several problems may occur using the above method. Considerable force is often required to push the tube through the tract because the surrounding tissues tend to contract immediately after the dilator is removed. The pyrex tube can fracture and the broken glass may be difficult or impossible to retrieve from the deeper tissues. The softer polyethylene tube tends to bend under the applied force and therefore may prevent the surgeon from being able to push the tube into place.

Other problems frequently occur early or late after surgery. The tube can migrate laterally or axially toward the eye as there is nothing to prevent this other than tissue contraction around the tube. This initates the eye or the tube can completely extrude. The tube may also migrate medially in spite of the flange. It can then become covered with conjunctiva or other tissue, and be impossible to reposition or sometimes to even locate.

Another potential problem can occur when the distal end of the tube lies against the nasal septum or other nasal tissues which block the distal opening of the tube so that tears cannot freely drain out the end of the tube. This prevents tears from the surface of the eye draining through and out the distal tube opening into the nose. As a result tears well up in the eye and run down the face. The patient constantly has to dab the eye. Some or all of the following procedures are required if the distal end of the tube is blocked. The tube can be removed and replaced with a shorter tube. However, this can only be performed if there is adequate room between the nasal septum and lateral nasal wall. Otherwise the tube will be too short to allow the distal end of the tube to extend beyond the lateral nasal wall. An alternative is to reposition the tube at a different angle. Repositioning alone is usually not sufficient. Both tube exchange and tube repositioning must typically be performed in the operating room. The third treatment is a nasal septoplasty if the nasal septum is deviated to the side of the tube. Again, this requires surgery in the operating room.

The diameter of the flange of the tube is selected to be large enough to discourage axial migration and conjunctival overgrowth while not being so large as to be unduly uncomfortable or prevent the flow of tears. This has resulted in a trade-off where overgrowth still occurs in some patients.

A pyrex tube has been proposed having a second smaller flange that is 4 mm from the main flange on the ocular surface end. However, this second flange makes the tube difficult to push into position, and even more difficult to reposition or replace. Therefore, it has rarely been used.

A canalicular bypass tube having large flanges on both the nasal and ocular ends has not been constructed because there would be no practical way to push it into position, and no practical way to extract it.

A pyrex bypass tube has been made having a hole through the flange for passage of a suture to temporarily attach to the surrounding conjunctiva. This feature only enhances axial stability while the suture is intact. Further, this approach also suffers from conjunctival overgrowth.

Therefore, a lacrimal bypass drainage device is needed which minimizes the above identified problems.

### SUMMARY

The instant embodiments provide a migration resistant lacrimal bypass drainage device. Some embodiments provide a lacrimal bypass drainage tube having an outer surface treated to provide for controlled axial friction and a flange on the ocular end. Axial friction is controlled by forming a helicoidal thread on the outer surface of the tube, thereby allowing the tube to be conveniently “screwed” into place, repositioned, or extracted. A removable biocompatible washer placed on the tube adjacent to the flange is provided to discourage tissue overgrowth immediately after emplacement. The flange is keyed to allow engagement by a tool having a correspondingly keyed surface to allow for the controlled application of torque.

In some embodiments, the preferred tool employs a keyed trocar portion secured to a manipulable handle. The trocar has a forward shaft portion for coaxially engaging the central cylindrical lumen of the drainage tube. In other embodiments, the shaft portion has a front end formed into a cutting bit and an opposite rear end formed into a radially widened haft. The haft caries two prongs which project axially forward to engage corresponding notches in the flange of the tube. The handle portion of the tool extends rearwardly from the haft. In one embodiment the cutting bit can be formed into two frustoconical symmetrically arcuate blades to enhance cutting during twisting manipulation of the tool. The keyed trocar can be made to be replaceable for differently sized, shaped or bladed trocars.

Some embodiments provide a drainage tube placed from the nasal side having a nasal side radial protrusions and a removable eye side flange in the form of a keyed nut. The nut is formed to have holes for engagement by temporary sutures immediately after emplacement. An overgrowth inhibiting biocompatible washer is provided to be placed on the tube adjacent to the nut.

Some embodiments provide a lacrimal bypass drainage device which comprises an oblong hollow tube defining a central lumen, and having first and second ends, and an outer surface; wherein said tube has a major axis and an axial dimension selected to span between the conjunctival cul-de-sac and the nose; a flange extending radially outward from a portion of said outer surface proximate to said first end; and, wherein said outer surface is shaped to have a threaded section. In some embodiments the threaded section is axially adjacent to said flange. In some embodiments the threaded section comprises a thread having a flattened crest-type shape. In some embodiments the flange is shaped to have a first angular bearing surface. In some embodiments the flange is shaped to have a first radial notch, thereby providing said first angular bearing surface. In some embodiments the flange is shaped to have a second radial notch diametrically opposite said first notch. In some embodiments the flange has a frustoconical outer surface and a substantially frustoconically shaped lumen entrance. In some embodiments a distal section of said outer surface tapers radially inwardly toward said second end. In some embodiments the tube is shaped to have at least one radial drainage opening spaced a distance from said second end. In some embodiments the tube is shaped to have at least one pair of radial drainage openings diametrically opposite from one another. In some embodiments the tube is shaped to have a plurality of radial drainage wherein a first of said plurality has a diameter greater than a diameter of a second of said plurality.

In some embodiments the device further comprises said threaded section being shaped to have a cross-section which exhibits defined mathematical derivatives at every concave part, and does not exhibit a defined mathematical derivative at a point in a convex part. In some embodiments the device is formed from a monolithic piece of material. In some embodiments the tube comprises PMMA. In some embodiments the tube has an axial length between said ends, said length being between about 5 millimeters and about 30 millimeters.

In some embodiments the device further comprises a washer having a central aperture sized and shaped to pass over said outer surface but not over said flange; and a peripheral edge portion sized to extend radially beyond a radial extent of said flange when said washer is mounted upon said tube. In some embodiments the washer has a non-planar shape. In some embodiments the washer has an outer diameter of between about 2.5 mm and about 15 mm.

In some embodiments, the device further comprises a keyed tool which comprises: a distal shaft sized to intimately penetrate said lumen; a proximal hand manipulable handle; and, a haft mounted between said shaft and said handle. In some embodiments the shaft terminates at a distal cutting bit. In some embodiments the device further comprises means for angularly securing said tube to said tool. In some embodiments the means comprise at least one prong extending axially from said haft. In some embodiments the flange is shaped to have a first angular bearing surface; and said haft is shaped to have a second angular bearing surface for bearing against said first angular bearing surface. In some embodiments the bit comprises a first blade. In some embodiments the blade has an arcuate cutting edge. In some embodiments the blade is axially arcuate. In some embodiments the bit further comprises a second blade diametrically symmetrical with said first blade. In some embodiments the tool further comprises an angular orientation indicator. In some embodiments the shaft comprises axial gradation markings.

Some embodiments provide that in a lacrimal bypass drainage device comprising a tube having an outer diameter first and second ends, and a flange extending radially outward from said outer diameter proximate to said second end, there is an improvement which comprises a biocompatible washer shaped and dimensioned to have a through-hole sized to accommodate the outer diameter of said tube; and a peripheral edge portion sized to extend radially beyond a radial extent of said flange when said washer is mounted upon said tube. In some embodiments the flange is shaped to have an angular bearing surface. In some embodiments the improvement further comprises means for resisting inadvertent axial movement of said tube. In some embodiments the means comprise said tube being shaped to have a helicoidal thread extending radially outwardly from said outer diameter.

Some embodiments provide a threaded lacrimal bypass cannula.

Some embodiments provide a kit for installing a bypass drainage tube in the body of a patient, said kit comprises: a first threaded cannula having a radially extending flange at a first end; said flange having a first angular bearing surface; and, a trocar having a second surface shaped and dimensioned to intimately contact and bear against said first surface when said trocar matingly engages said cannula. In some embodiments the first angular bearing surface defines a given cross-sectional geometry; and wherein said second surface has a cross-sectional geometry substantially symmetrical with said given cross-sectional geometry. In some embodiments the first cannula has a first axial length, and said kit further comprises a second cannula having a second axial length greater than said first axial length. In some embodiments the kit comprises a plurality of differently sized cannulas.

Some embodiments provide a trocar comprising a keyed haft. In some embodiments the trocar further comprises a handle secured to said haft. In some embodiments the trocar further comprises an angular orientation indicator.

Some embodiments provide a method for forming a lacrimal bypass drain which comprises: forming a tract between the conjunctiva and the nasal cavity of a patient; selecting an open ended hollow tube having a keyed flange at a first end and an opposite second end, and a threaded outer surface; and, emplacing said tube into said tract. In some embodiments the emplacing comprises: securing said tube to a trocar having a cross-sectional geometry sized and shaped to matingly engage said keyed flange; manipulating said trocar using said handle. In some embodiments the manipulating comprises: simultaneously axially pushing and angularly rotating said trocar. In some embodiments the method further comprises adjusting an axial position of said tube by rotating said tube.

Some embodiments provide that in a lacrimal bypass drainage device comprising a tube having an outer diameter, first and second ends, and a flange extending radially outward from said outer diameter proximate to said second end, an improvement which comprises said tube having an outer surface portion formed into a helicoidal thread.

### BRIEF DESCRIPTION OF THE DRAWINGS

FIG. 1 is a diagrammatic perspective view of the components of the lacrimal bypass drainage device.

FIG. 2 is a diagrammatic perspective view of the drainage cannula of FIG. 1.

FIG. 3 is a diagrammatic cross-sectional left side view of the cannula of FIG. 2 taken along line 3-3.

FIG. 4 is a diagrammatic cross-sectional side view of the cannula threads according to one embodiment of the invention.

FIG. 5 is a diagrammatic cross-sectional side view of buttress-shaped cannula threads according to an alternate embodiment.

FIG. 6 is a diagrammatic cross-sectional side view of hook-shaped cannula threads according to an alternate embodiment.

FIG. 7 is a diagrammatic ocular end view the cannula of FIG. 2.

FIG. 8 is a diagrammatic perspective view of the emplacement tool component of FIG. 1.

FIG. 9 is a diagrammatic cross-sectional left side view of the tool of FIG. 8 taken along line 9-9.

FIG. 10 is a diagrammatic cross-sectional top view of the tool of FIG. 8 taken along line 10-10.

FIG. 11 is a diagrammatic perspective view of the washer component of FIG. 1.

FIG. 12 is a diagrammatic cross-sectional left side view of the washer of FIG. 11 taken along line 12-12.

FIG. 13-FIG. 15 are diagrammatic views of the method steps for emplacing a lacrimal bypass drainage tube according to one embodiment of the invention.

FIG. 16 is a diagrammatic perspective view of the tube engaging portion of an adjustment tool according to an alternate embodiment.

FIG. 17 is a diagrammatic cross-sectional left side view of the tool of FIG. 16 taken along line 17-17.

FIG. 18 is a diagrammatic perspective view and zoomed view of the tube engaging trocar portion of an emplacement tool according to an alternate embodiment.

FIG. 19 is a diagrammatic left side elevational view of the cutting bit portion of the trocar of FIG. 18.

FIG. 20 is a diagrammatic distal end elevational view of the cutting bit portion of the trocar of FIG. 20.

FIG. 21 is a diagrammatic cross-sectional left side view of the cutting bit portion of a trocar according to an alternate embodiment.

FIG. 22a-FIG. 22k are diagrammatic ocular end views of various embodiments of a lacrimal bypass drainage tube providing angular bearing surfaces.

FIG. 23 is a diagrammatic perspective view of an alternate embodiment of a lacrimal drainage cannula having a conical flange and radial drainage opening.

FIG. 24 is a diagrammatic perspective view of an alternate embodiment of a lacrimal drainage cannula having a removable nut-type flange and dual opposite radial drainage openings.

FIG. 25 is a diagrammatic perspective view of an alternate embodiment of a lacrimal drainage cannula for emplacement from the nasal side.

FIG. 26a-FIG. 26h are diagrammatic views of the method steps for emplacing a lacrimal bypass drainage tube according to the cannula of FIG. 25.

### DESCRIPTION OF THE EXEMPLARY EMBODIMENTS

Referring now to the drawing, there is shown in FIG. 1, a first embodiment of the lacrimal bypass drainage device 1 according to the invention. The device includes a cannula or drainage tube 2, an overgrowth inhibiting washer 3, and a hand tool 4 for allowing emplacement and adjustment of the tube in a patient.

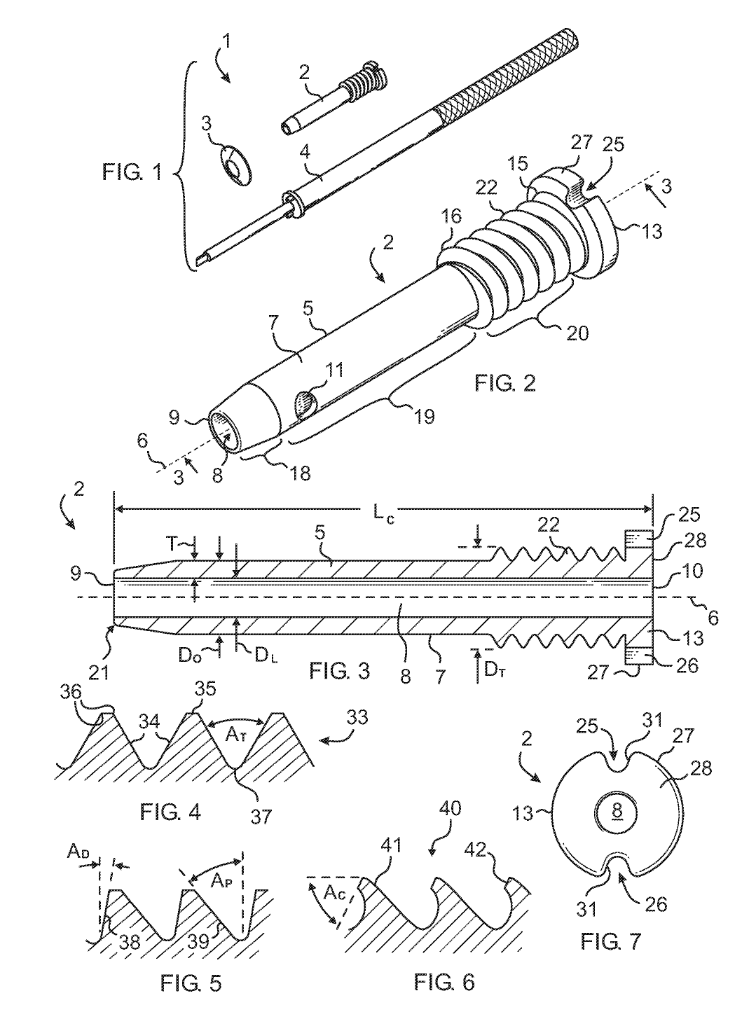

Referring now to FIGS. 2-3, the tube 2 is preferably made from an integrated, monolithic piece of fracture resistant, biocompatible material such as polymethylmethacrylate (PMMA), titanium or other rigid or semi-rigid durable material. The tube is shaped to have a generally oblong cylindrical body 5 having a central major axis 6, a cylindrical outer surface section 7 of a given outside diameter D_{O} and a cylindrical central axial lumen 8 of a given inside diameter D_{L} which is less than the outside diameter and extends from a first opening at a distal, nasal end 9 to a second opening at an opposite proximal, ocular end 10. The tube has a flange 13 located at the ocular end which extends circumferentially and radially outwardly beyond the outer surface of the body. The tube body has a medial section 19, a threaded section 20, and a distal tapered prow section 18 at the nasal end 9.

The tube has a given axial length L_{C} which is selected according the anatomy of the patient. For most human patients the length is preferably between about 5 millimeters (“mm”) and about 30 mm, and most typically between about 15 mm and about 22 mm. A number of specific length tubes can be made available as part of a kit so that the surgeon has a choice for a given situation. For example, a kit can contain six differently sized tubes ranging from 15 mm to 22 mm at 1 mm increments.

Similarly, the outside diameter D_{O} of the tube body in the medial section 19 is selected according to a patient's anatomy. A typical range in humans is between about 1 mm and 6 mm, and most typically is about 2.5 mm. The lumen diameter D_{L} is selected to provide adequate drainage throughput while maintaining structural soundness in the tube and is therefore dependent on the tube material as well as patient anatomy and condition. For a tube made from PMMA the inside diameter is selected so the thickness T of the tube wall in the median section is preferably at least 0.1 mm, more preferably at least 0.5 mm, and most preferably about 1.3 mm. Therefore, for most applications using a PMMA tube, the preferred range of the inside diameter is between about 0.25 mm and about 5 mm, and most typically is about 1.3 mm.

The distal prow section 18 of the tube has a given axial length and gradually tapers to form a generally frustoconical outer surface or otherwise tapered shape to facilitate emplacement. The axial length of the prow section preferably ranges between about 0.1 mm and about 2.5 mm, and most typically is about 2.2 mm. The outer diameter of the prow gradually tapers or decreases from the outside diameter D_{O} of the medial section to slightly greater than the lumen diameter D_{L} at the distal end 9 of the tube body so that a sharp edge is avoided at the distal end. Also, a rounded edge 21 is preferred to facilitate emplacement. For a tube having an outside diameter of 2.5 mm and a lumen diameter of 1.3 mm, the outer diameter of the prow goes from about 2.5 mm at its widest to about 1.4 mm at the nasal end. It should be noted that the outer diameter of the body may taper over part or all of the length of the tube.

A first radial drainage opening 11 is provided extending through from the outer surface of the tube body 5 to the lumen 8. In this way, tears drain into the nose through the radial opening when the nasal septum or other nasal tissue blocks the distal, axial opening of the tube at the distal end 9. The opening can be circular, elliptical, oval or other shape. Preferably the shape is rounded so that corners do not exist to trap fluid. The center of the opening is located proximate to but spaced apart a distance from the distal, nasal end 9 of the tube. This distance preferably ranges between about 1 mm and 15 mm and is typically about 2.5 mm. It is further preferable that the radial opening 11 does not extend axially into any tapered prow section 18 which could create surfaces impacting the insertability of the tube. The diameter of the radial opening preferably ranges between about 0.005 mm and 4 mm, and is typically about 0.5 mm, but will depend on the diameter of the tube, the tube material, its wall thickness, and the location of the radial opening or additional radial openings as described below. It should be noted that the tube can optionally be formed to have a rounded, closed distal end to allow easier insertion when no keyed trocar is used. In this case, one or more radial drainage openings would be required.

The tube 2 is formed to have a generally helicoidal threaded section 20 where at least one helicoidal thread 22 extends radially outwardly from the outside diameter D_{O} of the medial section to the outer diameter D_{T} at the thread crest. The threaded section allows the axial position of the tube to be adjusted by imparting a twisting motion upon the tube through application of sufficient torque to overcome the friction exerted by the surrounding tissue. The thread provides the tube with an angularly controlled radial prominence for discouraging inadvertent, unintended axial migration of the tube once it has been emplaced. The threaded section 20 preferably extends an axial length from a proximal part 15 adjacent to the flange 13 to a distal part 16 adjacent to the medial section 19 of the tube body 5. It should be noted that the threaded section need not contact the flange, but should be located to engage the walls of the tract formed between the conjunctiva and the nasal cavity. The axial length of the threaded portion is preferably between about 10% and about 100% of the total axial length of the tube. In most instances, it is preferable to have the medial section 19 of the tube having a smooth outer surface to facilitate penetration through the tissues, particularly at a part which passes through the lateral nasal wall. In most instances, the axial length of the threaded section ranges between about 1 mm and about 30 mm, and most typically is about 5.5 mm. In most instances, the inside diameter of the thread troughs or roots ranges between about 0.005 inch and about 0.89 inch. It should be noted that this diameter can be smaller than the outside diameter D_{O} of the tube body in the medial section but greater than the lumen diameter D_{L}. In most instances, the outside diameter D_{T} of the thread crests ranges between about 0.01 inch and about 0.5 inch.

The threaded section is preferably shaped and dimensioned for the unique purpose of permanently or semi-permanently engaging the soft tissue in the lacrimal zone to a degree which discourages or prevents axial migration but without unduly creating structures which are too large to be accommodated by the surrounding anatomy, or can trap fluids and lead to infection. This is in marked contrast to other surgical devices which may use threaded tubes for the relatively short duration of the operative and/or post-operative periods and which do not have the anatomical restrictions imposed by the lacrimal area. It is preferred that in most cases the thread will act to discourage axial migration without the need for additional structures such as sutures, will help to partially cut the tract as the tube is emplaced, and will not unduly lacerate tissues during intentional or unintended extraction. Although many types of thread cross-sections may work adequately, preferred cross-sections will address the above requirements in a superior manner.

In FIG. 3 the cross-section of the threads shows a curved shape having rounded crests and rounded troughs. The cross-sectional plane includes the central axis 6 of the tube. These smooth and rounded features discourage fluid collection which can lead to infection and avoid laceration after emplacement or during removal. Unfortunately, such a shape is less resistant to axial migration than thread shapes having a larger diameter or having a sharper cross-section, and will tend not to cut a tract during emplacement.

Referring to FIG. 4, there is shown the threaded section 33 of a bypass tube having threads which have a Unified National Coarse (“UNC”) or Unified National Fine (“UNF”) shaped cross-section. This cross-sectional thread shape is characterized by a series of generally equilateral triangles forming the faces 34 and forming a thread angle A_{T} of typically 60 degrees. A crest portion 35 of the thread is flattened to form defined corners 36 between the crest portion and either face to facilitate the cutting or tapping of a threaded tract during emplacement, but is not so sharp as to lacerate after emplacement or during removal. The root or trough 37 of the thread is rounded or otherwise shaped to provide a smoothed, curved concave surface to avoid fluid stagnation. In most instances, the thread angle A_{T} ranges between about 45 degrees and about 75 degrees, and is most typically about 60 degrees. In most instances, a thread pitch is selected to provide between about 1 crest per inch and about 80 crests per inch. Although many standard machine type threads having a flattened crest-type shape are acceptable, the preferred thread type is UNC 6-32 type threads. In most instances, other acceptable thread types vary from UNF 0-80 to UNC 5.875-16.

Referring to FIG. 5, there is shown an alternate embodiment wherein the threads have a Buttress-type cross-sectional shape to further discourage laceration during intended or unintended extraction. For this thread shape an angle A_{D} is formed between a distal face 38 and a plane perpendicular to the axis of the tube, and an angle A_{P} is formed between a proximal face 39 and a similar perpendicular plane. In most instances, angle A_{D} ranges between about 0 degrees and about 70 degrees and is most typically is about 10 degrees to provide adequate dwell for manufacturing economy, and angle A_{P} ranges between about 10 degrees and about 70 degrees, and is most typically is about 45 degrees.

Referring to FIG. 6, there is shown a second alternate embodiment wherein the threads have a hook-type cross-sectional shape 40 wherein the distal face 41 has a convex shape to further discourage laceration during extraction. Further, the flattened portion 42 of the crest is angled toward distal end of the tube forming an angle A_{C} with a plane parallel to the tube axis. In most instances, angle A_{C} ranges between about 1 degree and about 120 degrees and is most typically is about 45 degrees to provide corners oriented to facilitate cutting or tapping of a tract during emplacement.

It is important to note that in the above embodiments relating to FIGS. 4-6, the troughs are smooth and the crests have corners. In other words, the thread cross-section exhibits defined mathematical derivatives at every concave part of the curve, but does not exhibit a defined mathematical derivative at at least one point in the convex part of the curve.

Referring now to FIGS. 2, 3 and 7, the flange 13 is generally circularly shaped and angularly extends 360 degrees circumferentially around the ocular end of the tube to provide an axial bearing surface 31 at the ocular end to bear an axial pushing force from the tool. The flange diameter is selected to be small enough for comfort and adequate drainage but large enough to discourage overgrowth. Because of the thread, the flange can have a smaller outside diameter while still allowing the tube to exhibit an adequate axial migration resistance. When used in combination with the temporary overgrowth inhibiting washer, the flange can have its radially largest portion be as small as 110% of the outside diameter D_{O} of the medial section. In most instances, the diameter of the flange preferably ranges between about 3 mm and about 8 mm, and most typically is about 4.5 mm. The axial length of the flange preferably ranges between about 0.25 mm and about 3 mm, and most typically is about 1 mm. Although not shown, suture holes can be provided to extend axially through the flange.

Although during emplacement the surgeon can grasp the flange with a toothed forceps other tool to impart the twisting motion upon the tube to screw it into place or adjust its axial position, the present embodiment of the device provides at least one angular bearing surface on the tube sized, shaped and positioned to contact a corresponding surface on a torque inducing tool. In other words, the tube has a surface oriented to contact a corresponding surface on a twistingly manipulable tool such as a screwdriver to conveniently impart a twisting motion upon the tube.

Therefore, the tube is formed to have two diametrically opposite notches 25,26 extending radially inwardly from the circular outer periphery 27 of the flange 13 to provide the angular bearing surface 31 on any surfaces which are not tangent to any cylinder coaxial with the central axis 6 of the tube. In this embodiment, the notches are 180 degrees apart or diametrically opposite one another, sized shaped and located to be matingly engaged by corresponding prongs on the haft of the tool as described below. The notches are axially uniform having a generally bell-shaped contour and rounded edges to avoid sharp edges which could irritate surrounding tissues. Because the tube can be engaged by a finite number of angular orientations of the tool, the tube flange can said to be keyed. Furthermore, the tool would have a surface which is correspondingly keyed. In other words, the tool has a first cross-sectional geometry sized and shaped to matingly engage a second cross-sectional geometry of the keyed flange.

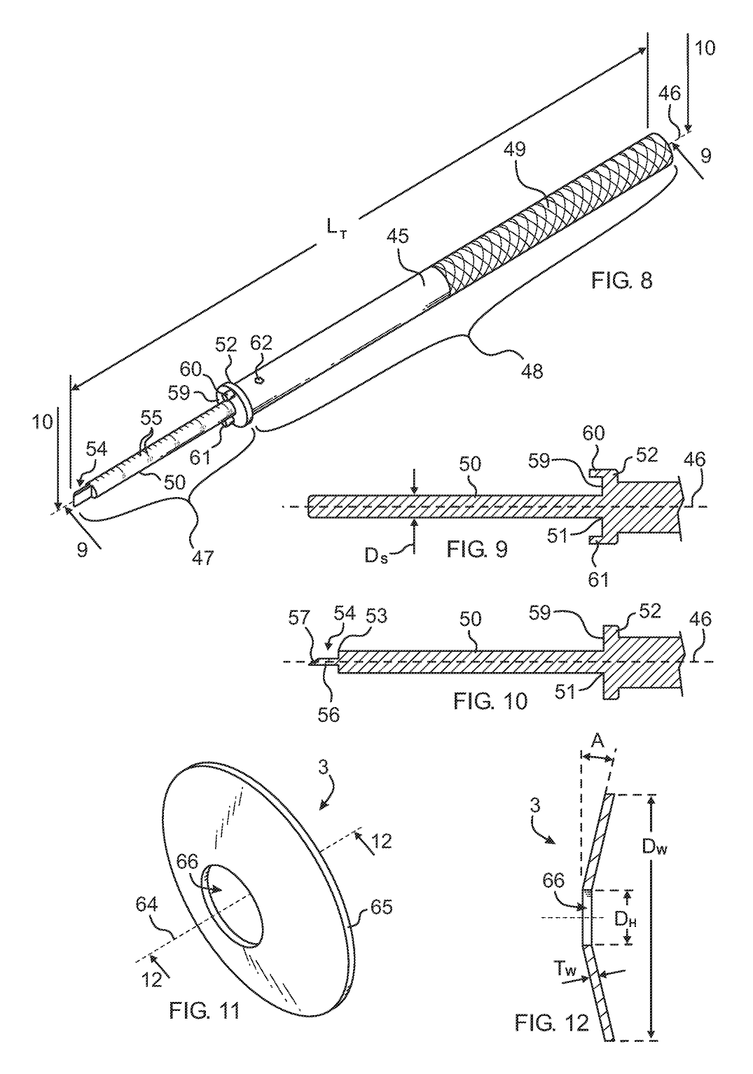

Referring now to FIGS. 8-10, the emplacement or adjustment tool 4 has a generally oblong body 45 having a major axis 46 and a rear or proximal handle portion 48 and a forward or distal tube engaging portion 47. The tool is preferably composed of a durable, easily sterilized material such as stainless steel. The tool has an axial length L_{T} which is preferably between about 25 mm and about 200 mm, and is typically about 127 mm. A knurled or otherwise roughened surface 49 is provided on the proximal 30 mm of the handle portion 48 to aid the surgeon in grasping and manipulating the tool. The roughened surface can extend the entire length of the handle portion. The handle portion has an outer diameter which preferably ranges between about 0.5 mm and about 10 mm, and is most typically 2.5 mm.

The tube engaging portion 47 has a substantially cylindrical oblong shaft 50 having a given outer diameter D_{S} sized to intimately engage the lumen of the tube. The rear or proximal end 51 of the shaft connects to a generally circularly shaped haft 52 which bonds to the handle portion 48 of the tool. The forward or distal end 53 of the shaft can be formed to support a cutting bit 54 such that the tube engaging portion forms a trocar. In most instances, the shaft has an axial length of between about 15 mm and about 22 mm from the axially proximal end of the bit to the axially distal end of the haft. This distance preferably matches the length of the lacrimal drainage tube. As such, a kit having a plurality of tools can be provided having shaft portions of different lengths corresponding to the different lengths of the drainage tubes provided in the kit. Optionally, the shaft may have axial gradations or other markings 55 which allow it to act as an axial measuring trocar to help ascertain or verify patient anatomy.

In this embodiment the cutting bit 54 of the trocar is formed by a single blade having a substantially planar tongue 56 terminating in a sharpened distal cutting edge 57. The blade has an axial length which is preferably between about 0.5 mm and about 10 mm, and most typically is about 2 mm. The distal cutting edge can be straight or curved, but typically is straight.

The haft 52 of the tube engaging portion is sized to matingly engage the flange 13 of the tube, and therefore extends radially beyond the outer diameter of the shaft and extends angularly 360 degrees circumferentially around the rear end of the shaft. This provides an axial bearing surface 59 for contacting the corresponding axial bearing surface 28 on the tube. The axial length of the haft preferably ranges between about 0.25 mm and about 5 mm, and most typically is about 1 mm. The diameter of the haft preferably ranges between about 3 mm to about 8 mm, and most typically is about 4.5 mm, but should not be so wide as to interfere with patient tissues during emplacement.

The haft 52 supports a pair of peripheral substantially cylindrical prongs 60,61 projecting distally and substantially parallel to and spaced apart from the shaft. The prongs are sized, shaped and located to matingly engage the notches 25,26 in the flange of the tube. Therefore, in this embodiment, the prongs are each approximately 0.8 mm in diameter and 1 mm in axial length, and are angularly spaced 180 degrees apart. The length of the prongs may vary from 0.25 mm to 5 mm.

The prongs thus angularly fix the lacrimal drainage tube to the tool and allow the lacrimal drainage tube to screwed or unscrewed by the tool. It should be noted that angular location of the prongs can be selected as an indicator of the orientation of the blade. This can be helpful to the surgeon when the blade is hidden from view, particularly when in use. Alternately, the orientation indicator can be an indicia 62 formed onto an outer surface at a specific angular location on the tool.

Referring now to FIGS. 11-12, a washer 3 is placed on the lacrimal drainage tube prior to screwing it into position. The washer prevents the lacrimal drainage tube from migrating medially and prevents overgrowth by the conjunctiva from covering the tube after surgery. The washer can be made from silicone, polyethylene, polyurethane, or other semi-rigid, resiliently flexible biocompatible materials. The washer is preferably disk shaped having a symmetrical axis 64 and a substantially circular outer circumferential edge 65 so that it will not appear to shift radially during axial rotation, having a preferred diameter D_{W} ranging between about 2.5 mm and 15 mm, and is typically about 7 mm. The washer has a substantially uniform thickness T_{W} which ranges from between about 0.005 inch and about 0.3 inch, and is typically about 0.02 inch thick. The thickness can also be selected to allow it to be easily cut and removed without repositioning the tube. The washer is shaped to have a central axial hole 66 having a diameter D_{H} commensurate with the outside diameter of the thread D_{T}, or more preferably, the outside diameter D_{O} of the lacrimal drainage tube so that the washer can be fastened to the tube by screwing it on. Therefore, the preferred diameter ranges between about 1 mm and 6 mm, and is typically about 2.5 mm. The washer can also be shaped to be non-planar to help prevent portions of the outer circumferential edge jutting into the surrounding tissue. Therefore, for example, the washer can have an arcuate, dish or, as shown in the drawing, a shallow conical shape providing a conical surface at an angle A a plane perpendicular to the axis 64.

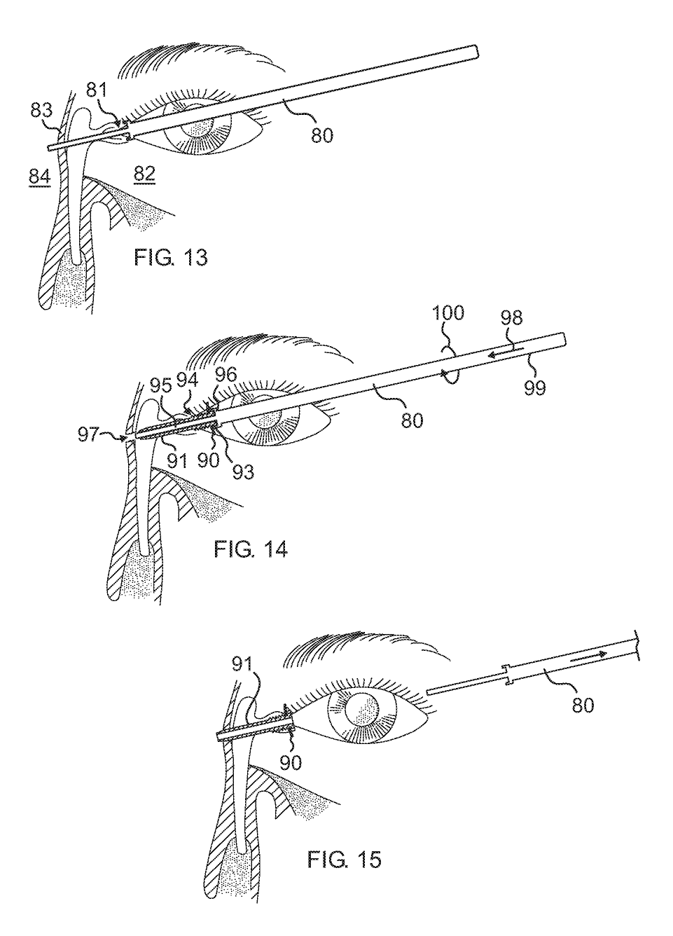

Referring now to FIGS. 13-15 the kit and device can be used as follows. Based on the patient anatomy, the surgeon selects the appropriate size of the lacrimal bypass drainage cannula or tube to be emplaced and a corresponding keyed trocar tool whether pointed, blunted, or bladed. As shown in FIG. 13, the trocar tool 80 (without the tube secured thereon) is pushed through the inferior caruncle 81 in the medial conjunctiva of the patient's eye area 82, through the intervening tissues including the lateral nasal wall 83 into the nose 84. The trocar alone is placed at this point to allow the formation of an initial tract, and to be sure that it is at the desired angle. The surgeon visualizes the distal tip of the trocar intranasally to confirm that it is properly angled. Optionally, the surgeon can use the trocar as a measuring device to determine or verify the optimal length of the tube. The trocar is then withdrawn. Optionally, the surgeon can first create a pilot hole using a needle prior to pushing the trocar through.

Referring now to FIG. 14, the washer 90 is placed over the lacrimal drainage tube 91 by inserting the distal end of the tube through the center hole of the washer until the washer is up against the flange 93. The tube is then mounted coaxially onto the shaft 95 of the trocar portion of the emplacement tool 80 so that the prongs on the haft 96 insert into the notches on the flange of the tube. Optionally, an amount of sterile, biocompatible lubricant such as MURILUBE brand mineral oil lubricant, commercially available from American Pharmaceutical Partners, Inc. of Schaumberg, Ill. can be used between the shaft and lumen to reduce friction and thus facilitate extraction of the trocar from the tube.

The trocar, with the mounted lacrimal drainage tube and washer, is then emplaced into the tract 97 that was previously created. This is done by first pushing the trocar with the lacrimal drainage tube and washer mounted thereon, medially 98 through the inferior caruncle in the medial conjunctiva until distal part of the threads 94 on the tube just contact the caruncle 81 in the medial conjunctiva. The surgeon now grasps the roughened proximal handle surface 99 of the tool and turns the tool clockwise 100 while gently pushing axially medially to screw the threaded end of the lacrimal drainage tube into the medial canthus. The lacrimal drainage tube is screwed in until the conjunctiva just touches the washer which is adjacent and medial to the flange of the lacrimal drainage tube.

Referring now to FIG. 15, the trocar tool 80 is removed. The lacrimal drainage tube 91 with the washer 90 is now emplaced. The washer can be trimmed with scissors if the surgeon deems this necessary. The tears can now freely drain through the lacrimal drainage tube into the nose. Later, the washer is incised with scissors and removed a few days to weeks after surgery. This is done after postoperative conjunctival edema and inflammation have resolved.

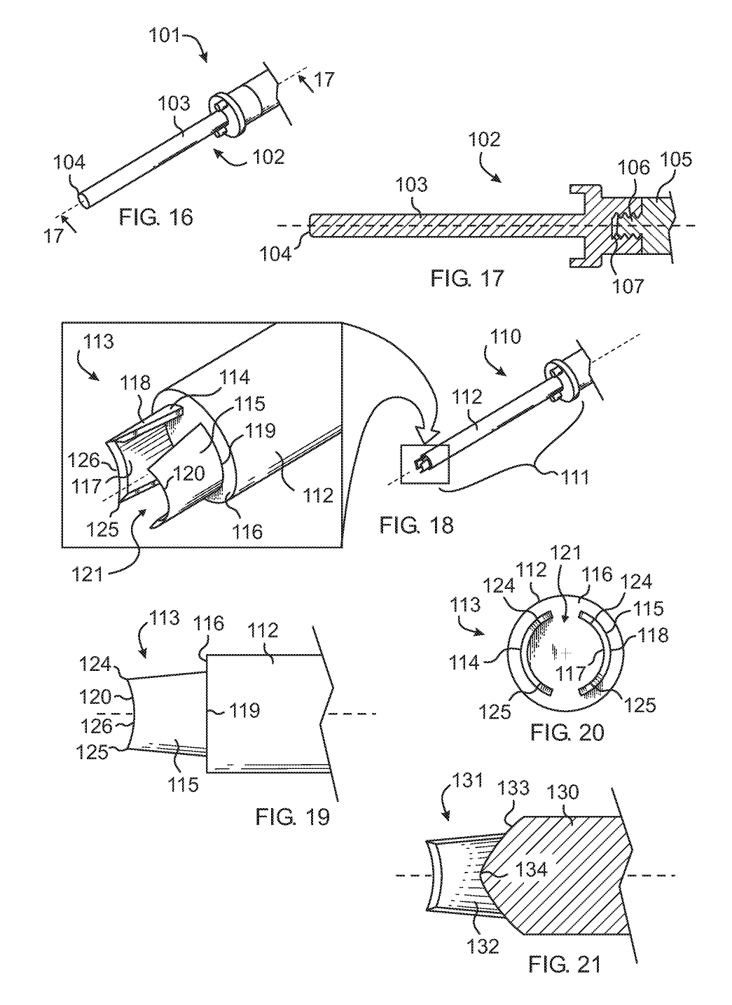

Referring to FIGS. 16-17, a non-bladed version of the tool 101 is provided in the kit to be used an adjustment tool after the lacrimal bypass drainage tube has been emplaced. The tool has a tube engaging portion 102 similar to previous embodiments, however, it has a shaft portion 103 which terminates in a blunted distal end tip 104. The adjustment tool can be used to screw or unscrew the lacrimal drainage tube and thereby adjust its axial positioning or remove it at any time during or after surgery.

Tube removal occurs as follows. The non-bladed version of the tool is inserted into the emplaced tube so that the keyed haft engages the correspondingly keyed flange on the tube. The surgeon then grasps the roughened handle surface of the tool and turns it counterclockwise while axially withdrawing the tool a corresponding amount. This unscrews the threaded end of the lacrimal drainage tube from the medial canthal tissues. When the threaded end of the tube has been entirely unscrewed from the medial canthal tissues, the surgeon removes the tool, and grasps the lacrimal drainage tube with a forceps and pulls it entirely out of the medial canthus, thus completely removing the tube.

This embodiment also provides a tube engaging portion 102 which is interchangeable by being releasably secured to the handle portion 105 of the tool using releaseable fastening means such as cooperatively threaded matable post and pit 106,107. It should be noted that this allows the kit to have a single handle onto which can be fastened a number of differently sized, shaped or bladed trocar-type tube engaging portions or non-bladed tube engaging portions.

Referring to FIGS. 18-20, a differently bladed trocar version of the tool 110 is provided in the kit which can more conveniently and predictably form the tract from the inferior caruncle to the nose using a simple axial rotation of the tool while pushing axially medially. Specifically, the tube engaging trocar portion 111 has a shaft 112 which terminates at a cutting bit 113. The cutting bit is formed to have a pair of blades 114,115 projecting distally from a distal end 116 of the shaft and diametrically separated from one another to define a central slot 121. Each blade is shaped to be generally semi-cylindrical, semi-conical, or otherwise arcuate having an inner concave surface 117 and an outer convex surface 118. The proximal end 119 bonds to the distal end of the shaft 112, and the distal end is beveled from the concave surface distally outwardly to form a cutting edge 120 at the convex surface. Further, the cutting edge is shaped to have an axially arcuate concave shape. In other words, depending on the elevational location, the cutting edge will have a different axial terminus. This shape provides the edge with a pair of angularly spaced apart points 124,125 located axially distally from an elevationally medial portion 126 of the blade. The two blades are preferably diametrically symmetrical to one another. In some situations where only soft tissue exists between the inferior caruncle and nasal cavity such as after a dacryocystrhinostomy has already been performed, this cutting bit embodiment allows for a single step emplacement of the lacrimal bypass drainage tube where the tract is opened simultaneously as the tube is screwed into place.

Referring now to FIG. 21, there is shown an alternate embodiment of the bladed trocar portion of the tool is provided in the kit which can more conveniently push tissue aside during formation of the tract. The trocar portion is similar to the embodiment of FIGS. 18-20 by providing a shaft 130 which terminates at a cutting bit 131 formed by a pair of arcuate blades (only one blade shown 132) projecting distally from a distal end 133 of the shaft. The distal end is formed to have a radially symmetric, convex, coaxial point 134 which helps tissue escape from between the blades through the central slots. The point may be sharp or blunted.

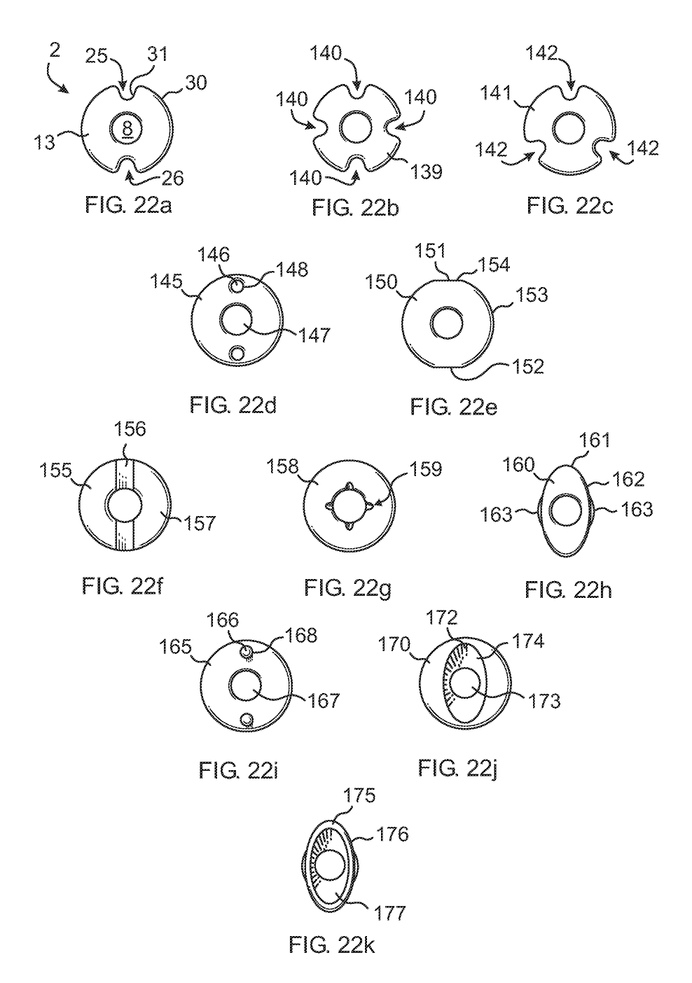

Referring now to FIGS. 22a-22k, there are shown variations in the shape and dimensions of the flange of the lacrimal bypass drainage tube which all still provide an angular bearing surface on any surfaces which are not tangent to any cylinder coaxial with the central axis of the tube, for the keyed engagement by the correspondingly shaped and dimensioned emplacement tool. The shape, dimensions, location, and number of prongs or other surfaces on the haft of the emplacement and adjustment tool can vary to correspond to the characteristics of the surfaces of the flange of the lacrimal drainage tube.

For reference, FIG. 22a shows the proximal ocular end view of a tube 2 according to the embodiment shown in FIGS. 1-3 having two notches 25,26 formed into the outer periphery 30 the flange 13 which are 180 degrees apart or diametrically opposite one another, sized shaped and located to be matingly engaged by the prongs on the haft of the tool. Each notch forms an angular bearing surface 31.

Alternately, FIG. 22b shows a tube flange 139 having four notches 140, and FIG. 22c shows a tube flange 141 having three notches 142 angularly spaced evenly apart to provide a greater number of angular orientations for the tube to be engaged and angularly secured upon the tool. A greater number of orientations can be beneficial if one notch gets obstructed temporarily.

Alternately, FIG. 22d shows a tube flange 145 having at least one axial bore 146 radially and parallelly spaced apart from the lumen 147, and providing an angular bearing surface 148.

Alternately, FIG. 22e shows a tube flange 150 having a pair of flattened facets 151,152 formed into the outer periphery 153 which are 180 degrees apart or diametrically opposite one another, sized shaped and located to be matingly engaged by flattened prongs, or a correspondingly shaped and dimensioned socket on the haft of the tool. Each facet forms an angular bearing surface 154.

Alternately, as shown in FIGS. 22f, the tube flange 155 can be formed to have groove 156 extending axially into and diametrically across the proximal surface 157 of the flange, which is analogous to a standard slot-headed screw fastener keyed engagement. In this way, a standard slot-head-type screwdriver tool can be used to adjust the tube.

Alternately, FIG. 22g shows a tube flange 158 having a standard Phillips-type keyed engagement 159. Those skilled in the art will readily appreciate other standard fastener keyed engagements such a hex or star-shaped engagement, for example.

Referring now to FIG. 22h, there is shown a tube flange 160 having an elliptically-shaped outer periphery 161 providing an angular bearing surface 162. It should be noted that the shape of the periphery can allow for some sections of it to have a smaller radial dimension than that of the crest of the thread 163. The haft of the tool can be formed to have a correspondingly shaped and sized socket for matingly engaging the flange.

Alternately, FIG. 22i shows a tube flange 165 having at least one axial bump 166 radially and parallelly spaced apart from the lumen 167, and providing an angular bearing surface 168.

Alternately, FIG. 22j shows a tube flange 170 having a substantially uniformly circular periphery 171 and a coaxial elliptically conical indentation 172 which narrows as it extends distally from the proximal end to the central lumen 173 thereby providing an axial bearing surface 174.

Alternately, FIG. 22k shows a tube flange 175 having an elliptically-shaped outer periphery 176 and an elliptically conical indentation 177 similar to that shown in FIG. 22j.

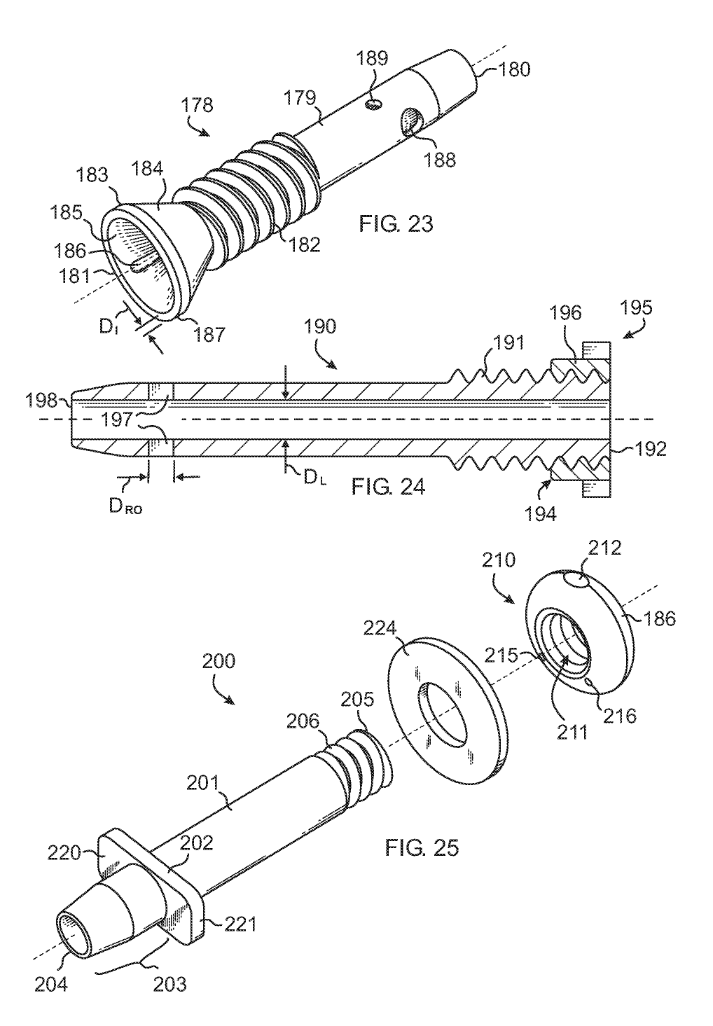

Referring now to FIG. 23, there is shown an alternate embodiment of the lacrimal bypass drainage tube 178 having a generally oblong cylindrical body 179 defining an axial lumen and terminating at a tapered distal, nasal end 180 and an opposite, proximal ocular end 181. A helicoidal thread having a flattened crest 182 is formed onto the outer surface of the body. A flange 183 extends radially outwardly and axially proximally from the ocular end and has a substantially frustoconical outer surface 184 and a substantially frustoconically shaped indentation or pit as an entrance 185 leading to the central lumen. An inwardly extending bump 186 provides an angular bearing surface to contacted by an emplacement tool. In some instances this type of flange can provide improved drainage by locating the edge of the indentation 185 within a closer distance D_{I} of the flange periphery 187 around the entire circumference of the flange to provide the same drainage capability regardless of angular orientation. First and second radial drainage openings 188,189 having different locations and diameters are shown to circumvent blockage of the axial opening at the distal end 180.

Referring now to FIG. 24, there is shown an alternate embodiment of the lacrimal bypass drainage tube 190 where the thread 191 extends to the ocular end 192 of the tube. A flange structure 195 is provided as a removable nut 196 which has an inner thread matingly corresponding to the thread of the tube. The nut has a rounded distal circumferential edge 194 for comfort and tissue irritation avoidance. The tube has a pair of radial openings 197 proximate to the distal, nasal end 198 of the tube and are diametrically opposite one another. In this way this insures that the surgeon, with slight rotation of the tube, can always be sure that at least one hole is oriented inferiorly to allow the best use of gravity to assist in tear drainage. Alternately, there can be a plurality of radial openings, which are particularly indicated in larger tubes or in situations where the location of potentially blocking radial tissues is uncertain.

Typically, the diameter of each opening D_{RO} would not be larger than the diameter of the tube's axial lumen D_{L}. Given these parameter ranges, the ratio between the diameters D_{RO}/D_{L} range from about 2% to about 80%, and more typically fall in the range of about 20% to 40% when a single pair of radial openings are used. When a plurality of openings are used such as five or more, the openings will obviously not all be opposite one another. In tubes having multiple radial openings, such as shown in the embodiment of FIG. 23, the diameters of the openings can be different depending on their distance from the distal end.

Referring now to FIG. 25, here is shown a further alternate embodiment of a surgically implantable lacrimal bypass drainage device providing a cannula or drainage tube 200 having axial migration inhibiting structures on both ends when emplaced. The device has an oblong tubular body 201 having a rounded cornered quadrangular end stop 202 spaced a distance 203 from a first nasal end 204 and an opposite ocular end 205 having a threaded section 206 sized to be engaged by a nut 210 having a substantially toroidal shape, defining an internally threaded axial hole 211, and having top and bottom flat surfaces or facets 212 provide an angular bearing surface for engagement by a hemostat or other implement which causes twisting of the nut onto, or off of the body. Two parallelly spaced axial suture holes 215,216 are formed through the nut and angularly spaced apart by an angle of about 45 degrees. The end stop structure 202 on the nasal end is a pair of radial protrusions 220,221 or “wings” and is integral with the tube. The migration inhibiting structure on the ocular end is the threaded nut 210 which extends circumferentially 360 degrees around the end of the tube when fastened thereon. The body and nut are each preferably made from an integrated, monolithic piece of fracture resistant, biocompatible material such as polymethylmethacrylate (PMMA), titanium or other rigid or semi-rigid durable material. A washer 224 is also provided and made of a semi-rigid or resiliently flexible biocompatible material such as silicone.

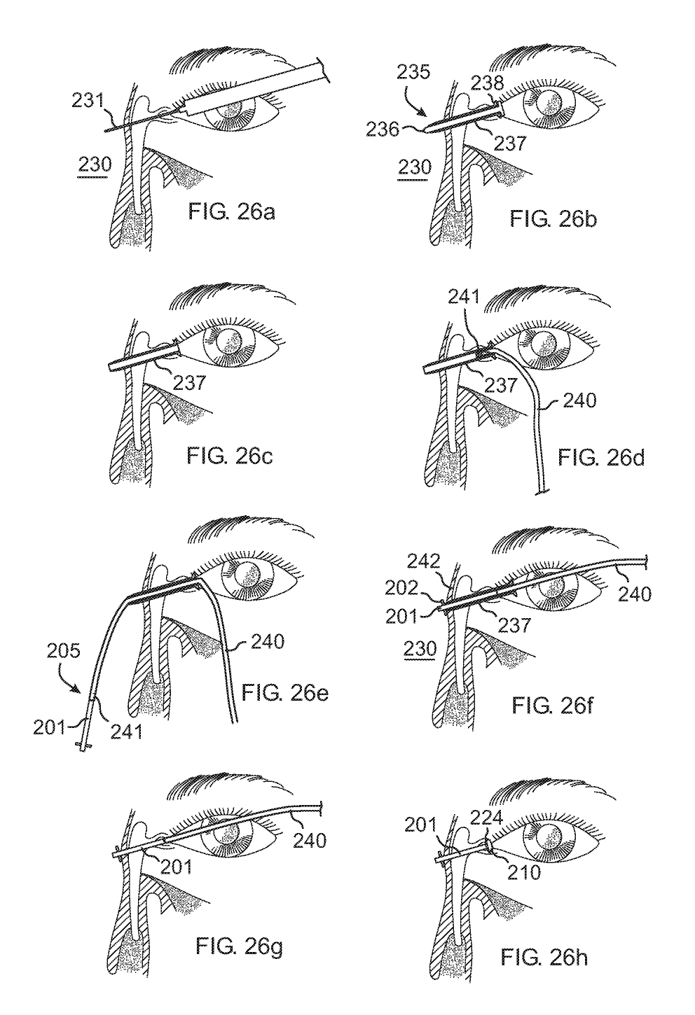

Referring now to FIG. 26a-26h, a surgical method for placement of the drainage tube of the embodiment shown in FIG. 25 is envisioned. In FIG. 26a, an initial opening is created by pushing a 16 gauge metal needle 231 from the conjunctiva of the medial canthus into the nasal cavity 230. This allows the surgeon to visualize the needle intranasally to be sure that the tract is properly angled. The needle is then withdrawn.

In FIG. 26b, and a steel trocar 235 is placed through the same tract from the conjunctiva through the intervening tissues into the nasal cavity 230. The trocar 235 has a substantially nail-shaped pin 236 and an outer metal sheath 237 having a flange on its proximal ocular end 238.

In FIG. 26c, the pin is removed leaving the outer sheath 237 in place.

In FIG. 26d, a hollow flexible positioning cable 240 made from sterile silicone rubber and having an internally threaded distal end 241 is inserted into the central axial lumen of the sheath 237 at its ocular end and through to the nasal cavity 230. The surgeon reaches up the nose and grasps the cable's distal end 241 with a hemostat and pulls it down the nose and out the naris. The cable now extends from the ocular surface through the trocar sheath down the nose and out the naris. In FIG. 26e, the proximal threaded end 205 of the drainage tube body 201 is then threaded into the distal end 241 of the cable 240.

In FIG. 26f, the cable 240 is slowly withdrawn, causing the body 201 of the drainage tube to be drawn up into the sheath 237. Care is taken to properly orient the drainage tube as it is being drawn through the nasal cavity 230 into the sheath. As the tube reaches the sheath, the end stop 202 bears against the distal end of the sheath. Further withdrawing of the cable causes the sheath to move laterally until the end stop bears against the lateral nasal wall 242.

In FIG. 26g, the sheath has been removed off the proximal end of the flexible positioning cable 240 leaving the body 201 of the drainage tube in the tract. The cable 240 itself is then detached from the body 201 of the drainage tube.

In FIG. 26h, the washer 224 and nut 210 are placed onto the thread of the drainage tube body 201 to emplace the drainage tube. The nut is manipulated using a hemostat or other tool. The drainage tube may then be further secured in place during the immediate postoperative period by the placement of sutures through the holes in the nut. This also allows the tube to be pulled proximally if needed.

While the preferred embodiments of the invention have been described, modifications can be made and other embodiments may be devised without departing from the spirit of the invention and the scope of the appended claims.

What is claimed is:

1.-22. (canceled)

23. A method for forming a lacrimal bypass drain comprises:
forming a tract between the conjunctiva and the nasal cavity of a patient;
selecting an open ended hollow tube having a keyed flange at a first end and an opposite second end, and a threaded outer surface; and,
emplacing said tube into said tract.

24. The method of claim 23, wherein said emplacing comprises:
securing said tube to a trocar having a cross-sectional geometry sized and shaped to matingly engage said keyed flange, and a handle;
rotationally manipulating said trocar using a handle to screwingly insert said tube said tract.

25. The method of claim 24, wherein said manipulating comprises:
simultaneously axially pushing and angularly rotating said trocar.

26. The method of claim 23, which further comprises:
adjusting an axial position of said tube by rotating said tube.

27. (canceled)

28. The method of claim 23, which further comprises:
repositioning said tube within said tract after said emplacement.

29. The method of claim 23, wherein said forming a tract and emplacing said tube occur simultaneously.

30. The method of claim 23, which further comprises:
removing said tube, wherein said removing comprises:
inserting a non-bladed tool into said tube wherein said tool has a cross-sectional geometry sized and shaped to matingly engage said keyed flange, and a handle; and
rotationally manipulating said tool using said handle to unscrewingly extract said tube from said tract.
